(19) United States
(12) Patent Application Publication
(10) Pub. No.: US 2003/0194427 A1
(43) Pub. Date: Oct. 16, 2003

(54) System of treating myopathic & neuropathic diseases

(76) Inventor: Anuthep Benja-Athon, New York, NY (US)

Correspondence Address: ANUTHEP BENJA-ATHON, M.D., Pain Management, 210 East 36th Street, New York, NY, 10016, US

(21) Appl. No.: 10/122,166

(22) Filed: Apr. 15, 2002

(51) Int. Cl. A61K 39/08; A61K 9/70; A61N 1/30

(52) U.S. Cl. 424/449; 604/20; 424/239.1

(57) ABSTRACT

A system of treating myopathic and neuropathic diseases comprises the delivery of neurotoxins to the muscles and nerves in the peripheral nervous and central nervous systems. The delivery of the neurotoxins is into or through the skin in human or animal. The system utilizes the uptake system, anterograde and retrograde axonal transports of neurons in the aforementioned nervous systems. The delivery system comprises of emulsifier, skin patch and jet injection. The chemicals are the amino acids, polypeptides, neurotoxins such as the botulinum toxins and venoms.

### FIELD OF INVENTION

A system comprises three methods of delivering therapeutic neurotoxins, myotoxins and venoms to an organ system in the treatment of various diseases in human and animal.

### BACKGROUND OF THE INVENTION

The first objective of the present invention is to provide a system for treating many diseases whose effective treatments are not known.

The second objective of the present invention is to provide a system for treating diseases related to the disorders and disorders of an organ system in a human or animal.

Specifically, the objective of the present invention is to provide a system for treating diseases and disorders of the peripheral nervous, central nervous and muscular systems.

More specifically, the objective of the present invention is to provide a system for treating muscular diseases, neuropathy, neuropathic disorders including pains, neuralgia, and itches.

The third objective of the present invention is to provide the best means of effectively and safely treating said diseases and disorders.

Many diseases and disorders afflict the muscular, peripheral and central nervous systems in human and animal for which there is no effective or no treatment. Said diseases and disorders are extremely painful and disabiling in patients and animals. The reasons are that the pathogenesis, mechanisms, effective means and method for treating said diseases and disorders are either poorly understood by most physicians and scientists, unknown or unavailable. As a result, untold suffering, torment, increased morbidity and mortality in human and animal are the rule. These patient and nation must expend billions of dollars per annum on ineffective treatments and devices.

Said diseases and disorders can be caused by genetic, metabolic or hormonal disorders, infections, trauma, etc. For example, in muscular system, the dystonias, tremors, involutary movements and spasm cause untold emotional and physical pains, suffering and disability.

Collorary, in the nervous system, the neuropathies, neuropathic pains, neuralgia and associated disorders including alteration of sensations and unbearable pains such as, but not limited to, allodynia, hyperalgesia, hyperesthesia and hyperpathia are a cruel torment man can ever experience. These are commonly seen in patients suffering from sympathetic nerve disorders, postherpetic neuralgia, diabetic neuropathy, itches, etc.

Briefly, further to same, in the muscular system, there are defects in the receptors or the make-up of the diseased muscles. In the nervous systems, damage or injuries to the neurons in the peripheral nervous system (PNS) or central nervous system (CNS) result in the sprouting of axons and/or dendrites of either damaged or undamaged but involved neurons. The axonal or dendritic collaterals from said sproutings lead to the formation of aberrant or modified neural circuits in PNS and/or CNS. In addition, there may be an activation, deactivation, disinhibition of various neural circuits. A myriad of neural chemicals such as hormones, neurotransmitters, amino acids, peptides and proteins are involved in said processes. Some common chemicals are such as, but not limited to, acetylcholine, substance P, glutamate, gamma-amino-butyric acid and calcitonin gene-related peptide.

In the treatment of many of said diseases and disorders, the present invention serves to provide a system of altering, affecting or modulating the organs described herein. In particular, the present invention serves as a model by using the system described infra to alter, affect and modulate the muscle and nerve by the introduction of an exogenous chemical at their receptors, membranes, and neural endings. A result is the alteration, affecting or modulation of the chemicals within said structures including various organelles in PNS and CNS. Therefore, the function and involvement of said neurons or organs in many diseases and disorders including those not mentioned herein are treated or modulated. The same application is used for other organs.

The present invention uses the venoms and neurotoxins including those not mentioned herein to achieve the goals setforth herein.

### SUMMARY OF THE INVENTION

Many Myopathic and Neuropathic Diseases Cannot be Treated.

The present invention is a system of treating myopathic and neuropathic diseases comprises the delivery of neurotoxins to the muscles and nerves in the peripheral nervous and central nervous systems. The delivery of the neurotoxins is into or through the skin in human or animal. The system utilizes the uptake system, anterograde and retrograde axonal transports of neurons in the aforementioned nervous systems. The delivery system comprises of emulsifier, skin patch and jet injection. The chemicals are the amino acids, polypeptides, neurotoxins such as the botulinum toxins and venoms.

### BRIEF DESCRIPTION OF THE DRAWINGS

FIG. 1 is the schematic illustration of the present invention.

FIG. 2 is the schematic detail illustration of the present invention.

### DESCRIPTION OF THE PREFERRED EMBODIMENTS

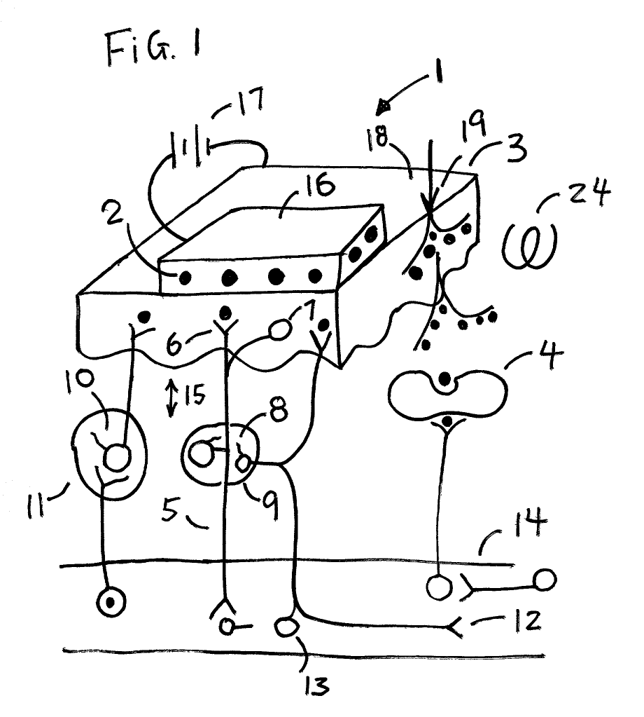

FIG. 1 shows the system 1 of the present invention. System 1 comprises three methods of delivery or deposition of exogenous therapeutic chemicals 2 (ETC) as represented by dark dots into the skin 3 and other organs 4 within skin 3 such as blood vessels or deep to skin 3 such as muscles, glands, soft and bony tissues in a human or an animal.

In skin 3 or other organs 4 within skin 3 or deep to skin 3, there are afferent and efferent neurons 5. Neurons 5 are sensory, autonomic and motor nerves having the first neural endings and 6 and receptors 7 in the skin, organs 4 within or deep to skin 3 such as muscular, visceral and pelvic organs.

Going toward the central nervous system, neurons 5 have the second neural endings, receptors, axons, dendrites and cell bodies 8 in the afferent nerve ganglion 9 and same 10 in the autonomic nerve ganglion 11 in the peripheral nervous system (PNS) and endings 12, presynaptic and postsynaptic receptors, axons and dendrites 13 in the central nervous system (CNS) 14. At the ganglion, spinal cord and brain levels, endings, receptors, axons and dendrites 8, 10, 12 and 13 form numerous and extensive neural connections.

In PNS and CNS, various endogenous chemicals and their precursors and substrates such as enzymes, neurohormones and neurotransmitters are synthesized, delivered, transported, released, uptaken and destroyed at the cell body, presynaptic and postsynaptic sites of all neurons as represented by neurons 5. The commonly known neurohormones and neurotransmitters are such as, but not limited to, acetylcholine, adrenaline and substance P. Said processes in neurons 5 in PNS and CNS involve the uptake and release mechanism and synthesis of the neural membrane and organelles of neurons 5. In addition, there are anterograde and retrograde axonal transport systems for transporting said neurochemicals away and towards the cell bodies as represented by a single double-arrow 15.

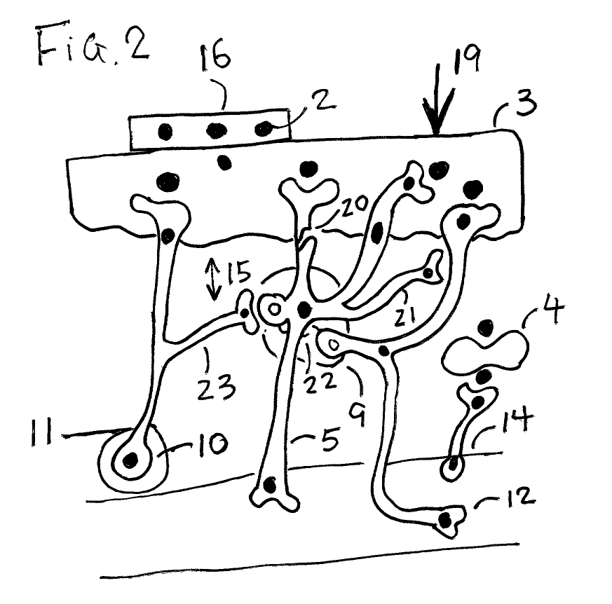

As described supra, in certain diseases and pains, certain neuronal population is damaged and/or destroyed as represented at 20 in FIG. 2. As shown in FIG. 2, the surviving neurons or affected neurons can regenerate collateral axons 21 or dendrites 22. Regeneration of said neurons involves the sprouting of axonal and dendritic collaterals similar to a cut tree sprouting new branches. Moreover, related or adjacent uninjured or undamaged neurons can also sprout axonal 23 and dendritic collaterals in response to signal from said affected neurons. A result is the formation of aberrant or abnormal connections and circuits. In addition, the chemicals in these neurons damaged or otherwise and related organs such as, but not limited to, enzymes, neurohormones and neurotransmitters are affected, synthesized, released or redeployed. A result is the manifestation of various types of diseases and pains in affected patients.

System 1 offers therapeutics for treating many diseases and illnesses in human and animal that to date have no effective treatment. System 1 effectively delivers exogenous therapeutic chemicals 2 to neurons 5 in skin 3 and organ 4 within or deep to skin. Using three methods described infra and said anterograde and retrograde axonal transport systems, ETC 2 can be delivered and introduced to neurons 5 and other organs 4 in skin 3 and be disseminated to the target neurons, or organs, in said locations in PNS and CNS as represents by dark dots in FIG. 2.

ETC 2 are such as, but not limited to amino acids, peptides, polypeptides and proteins including venoms and neurotoxins can be delivered and uptaken by the endings and receptors of neuron 5 or other organs in skin 3. These local sites of introductions and local target sites are neural receptors, endings, presynaptic site and postsynaptic site of neurons 5 directly or indirectly affected by ETC 2. In other words, the intended effect of ETC 2 may be local in neurons 5 in skin 3 or organs 4.

Further from the sites of introduction of ETC2, the other internded effect of ETC 2 is for the anterograde or retrograde transport system of neurons 5 to transport chemicals 2 and/or their derivatives away from the skin 3 or organs 4 proximally toward neurons 5's proximal components such as the cell bodies wherein lie various organelles, other neurons in ganglia and 11, spinal cord and brain.

Examples of ETC 2 are such as, but not limited to, venoms such as those of the insects, arthropods, reptiles and fish such as, but not limited to, bee, yellow-jacket, spiders and snakes. And neurotoxins such as, but not limited to, botulinum toxins and bungarotoxins. Commercial preparations of botulinum toxins are available from various companies including Allergan, Inc.

In all, ETC 2 not only alter, affect, modulate the neurochemistry of neurons 5 at the sites of introduction and uptake, but ETC 2 and/or their derivatives also reach targeted sites proximally such as receptors, endings, presynaptic site, postsynaptic site, cell bodies and their organelles, and other neurons having dendrites, axons and cell bodies in ganglia 10 and 11 in PNS and CNS.

Not shown in FIG. 1, the pharmacokinetics of ETC 2 in the first method is to allow ETC 2 to diffuse down a chemical gradient into skin 3 to neurons 5 and organs 4. In this setting, ETC2 can be mixed with and suspended in a water, oil or water-oil emulsifier, ge or cream. Said mixture can be applied to or rubbed into skin 3.

Alternatively, as an extension of said method and pharmacokinetics, said mixture preparation can be incorporated into a skin patch 16 having skin adhesive which then behaves like an occlusive dressing. Features of control-timed release of ETC 2 can also be incorporated into said designs.

To facilitate the penetration of ETC 2 of the superficial layers of skin 3 to reach ETC 2's targeted neurons 5 and organs 4, a means for increasing the permeability or porosity of skin 3 to ETC 2 can be used such as, but not limited to, occlusive dressing represents by a component of said skin patch and chemical substances such as, but not limited to, DMSO.

In the second method 17 of delivery ETC 2 as shown in FIG. 2, in addition said method, electrical current being produced by an electricity source can be used to drive ETC2 into skin 3 and to organs 4.

In the third method 18 of delivery ETC 2 to neurons 5 in skin 3, organs 4 in skin 3 or deep to skin 3 and other neurons proximal and deep to neurons 5 in skin 3, high-velocity, needleless jet injection as represented by a bold, single arrow 19 can effectively and widely dispersed ETC 2 to the targeted neurons 5 in skin 3 and organs 4 in or deep to skin 3. In the spirit of the present invention, the quantity, titration, various doses and concentrations of ETC 2 can be best controlled. This method allows ETC 2 to be effectively, selectively and least-expensively used to target neuron 5 or organs 4 within or deep to skin 3. In other words, the introduction, delivery and the pharmacokinetics of ETC 2 can be regulated or controlled by an injector or a physician. Said instrument in different models to be used by the present invention is made by various companies including Bioject, Inc. in Portland, Oreg., www.bioject.com.

Upon or during the introduction or movement of ETC 2 using said methods, electric field or magnetic field can be used as a drive to guide said chemicals to said target site as represented by a coil 24 in FIG. 1. This is facilitated by modern equipment including related and to-be-derived technology such as, but not limited to, diagnostic ultrasound, CTScan and MRI. The target sites are such as, but not limited to, receptor site in various organs, neoplastics including nerve tumors or cancers, neuromuscular site, nerve ending or end plate, ganglion, motor point, nerve fiber and tract in the PNS and CNS. This is made possible and preferred as said chemicals can be or made to become electrically charged.

Although various preferred embodiments and methods of this invention have been described, it will be appreciated by those skilled in the art that adaptations, variations and further incorporation of other methods and technology may be made without departing from the spirit of the invention and the scope of the claims.

Although the preferred embodiments of the present invention includes amino acids, polypeptides, proteins, neurotoxins, myotoxins and venoms, it will be appreciated by those skilled in the art that other chemicals not belonging to said classess including nonbiological and synthetic chemicals such as, but not limited to, various concentrations of aqueous phenol in water or oil, alcohols and combinations thereof may be applied and used without departing from the spirit of the present invention and and the scope of the claims.

1. A system of delivering means for altering the neurochemistry of the neurons in the peripheral nervous system and central nervous system of a human and an animal wherein said neurons have the first neural endings and receptors in the skin or in a first organ system of said human and animal, wherein said neurons have the second neural endings and receptors, axons and dendrites in the ganglion and central nervous system, and wherein said method for delivering in said neurons utilizes the uptake system, anterograde and retrograde axonal transports of said neurons comprises:
means for altering the neurochemistry of the neurons in the peripheral nervous system and central nervous system;
means for altering the neurochemistry of neurons at the neural endings, presynaptic site and postsynaptic site;
means for increasing the permeability of the skin to said means means for altering said neurochemistry;
means for delivering said means for altering the neurochemistry into said skin;
means for delivering said means for altering the neurochemistry through said skin;
electro-magnetic means for guiding and driving said means for altering the neurochemistry to said target site in PNS and CNS; and
a means for delivering said means for altering the neurochemistry into a first organ system.

2. The system of delivering according to claim 1 comprises said means for altering the neurochemistry of the neurons, an emulsifier means for retaining said means for altering the neurochemistry, and an emulsifier means for releasing said means for altering the neurochemistry.

3. The system of delivering according to claim 1 comprises a skin patch means for delivering said receptor means for modulating the function of a first organ system.

4. The skin patch according to claim 4 comprises said means for altering the neurochemistry, an emulsifier means for retaining said means for altering the neurochemistry, and an emulsifier means for releasing said means for altering the neurochemistry by diffusion down a chemical gradient into said skin to said first neural endings and receptors.

5. The skin patch according to claim 4 comprises said means for altering the neurochemistry, a gel means for holding said means for altering the neurochemistry, a gel means for releasing said means for altering the neurochemistry by diffusion down a chemical gradient into said skin, and an adhesive means for permeating said means for altering the neurochemistry into said skin to said first neural endings and receptors.

6. The system of delivering according to claim 1 comprises said means for altering the neurochemistry in an emulsifier of a skin patch through which electrical current is applied to exert an electronic force means for driving said means for altering the neurochemistry into said skin to said first neural endings and receptors.

7. The system for delivering according to claim 1 is a high-velocity jet injection means for delivering said means for altering the neurochemistry into said skin to said first neural endings and receptors.

8. The system for delivering according to claim 1 is a high-velocity jet injection means for delivering said means for altering the neurochemistry into said first organ system.

9. The system for delivering according to claim 1 is a high-velocity jet injection means for delivering said means for altering the neurochemistry to said first neural endings and receptors.

10. The means for altering the neurochemistry according to claim 1 are the amino acids.

11. The mean for altering the neurochemistry according to claim 1 are the polypeptides.

12. The means for altering the neurochemistry according to claim 1 are the neurotoxins.

13. The neurotoxins according to claim 16 are the botulinum toxins.

14. The neurotoxins according to claim 16 are the venoms.

15. The system of delivering according to claim 1 comprises a skin patch means for delivering the neurtoxins.

16. The system of delivering according to claim 1 comprises a skin patch means for delivering the botulinum toxins.

17. The system for delivering according to claim 1 is a high-velocity jet injection means for delivering the neurotoxins.

18. The system for delivering according to claim 1 is a high-velocity jet injection means for delivering the botulinum toxins.

19. The system for delivering according to claim 1 is a high-velocity jet injection means for delivering the venoms.

20. A system of of delivering means for modulating the neurotransmitters and the function of the organelles in the neurons, presynaptic and postsynaptic sites in the peripheral nervous system and central nervous system that are involved in neuropathy, neuropathic disorders including pains, itches in a human and an animal wherein members of said neurons have the first endings and receptors in the skin and a first organ system of said human and animal, wherein said neurons have the second endings, axons, dendrites and cell bodies in the ganglia, peripheral and central nervous systems, and wherein said means for delivering utilizes the uptake system, anterograde and retrograde axonal transports of said neurons comprises:
means for altering the neurochemistry of the neurons in the peripheral nervous system and central nervous system;
means for altering the neurochemistry of neurons at the neural endings, presynaptic site and postsynaptic site;
means for increasing the permeability of the skin to said means means for altering said neurochemistry;
means for delivering said means for altering the neurochemistry into said skin;
means for delivering said means for altering the neurochemistry through said skin;
electromagnetic means for guiding and driving said means for altering the neurochemistry to said target site in PNS and CNS; and
a means for delivering said means for altering the neurochemistry into a fist organ system.
